(19) United States
(12) Patent Application Publication
(10) Pub. No.: US 2023/0330029 A1
(43) Pub. Date: Oct. 19, 2023

(54) RESVERATROL COMPOSITION, PREPARATION METHOD AND APPLICATION THEREOF

(71) Applicants: CHENLAND NUTRITONALS INC, Irvine, CA (US); QINGDAO CHENLAND HEALTH INDUSTRY GROUP CO., LTD, Qingdao (CN)

(72) Inventors: SHENGCAN ZOU, Qingdao (CN); Jie Wang, Qingdao (CN); Xin Li, Qingdao (CN); Li Li, Qingdao (CN); Shanglong Wang, Qingdao (CN); Bin Han, Qingdao (CN); Liang Lin, Qingdao (CN)

(21) Appl. No.: 17/929,320

(22) Filed: Sep. 2, 2022

(30) Foreign Application Priority Data
Apr. 14, 2022 (CN) 202210393208.9

(51) Int. Cl. A61K 9/48; A61K 31/05; A61K 9/14; A61J 3/02; A61K 9/50

(52) U.S. Cl. CPC A61K 9/4866; A61K 31/05; A61K 9/146; A61J 3/02; A61K 9/5089

(57) ABSTRACT

A resveratrol composition and its preparation method and application are disclosed, which relates to the technical field of resveratrol solid dispersion preparation. A resveratrol composition is prepared from following raw materials: 6%-60% by weight of resveratrol, and 40%-94% by weight of poloxamer. The disclosure mixes the resveratrol and poloxamer in proportion to prepare a composition, and further improves the dissolution and bioavailability of the resveratrol in combination with microencapsulation technology. The preparation process is suitable for industrialization and has good application and promotion prospects.

### CROSS REFERENCE TO RELATED APPLICATION

This patent application claims the benefit and priority of Chinese Patent Application No.202210393208.9 filed on Apr. 14, 2022, the disclosure of which is incorporated by reference herein in its entirety as part of the present application.

### TECHNICAL FIELD

The present disclosure relates to the technical field of resveratrol solid dispersion preparation, and more specifically, to a resveratrol composition and its preparation method and application.

### BACKGROUND ART

Resveratrol is found in many common Chinese medicines such as Polygonum cuspidatum, Veratrum nigrum and Cassia obtusifolia, as well as grapes, peanuts and other foods. It has been widely used to treat cardiovascular diseases, arteriosclerosis, hyperlipidemia and other diseases. It can also be used as a food additive and made into health products. In the world, the research on it is mostly limited to the protection mechanism, pharmacological action, cyclodextrin inclusion process, etc. There are few reports on the preparation process of new solid dispersion to improve its solubility, and no finished product has been put on the market.

Resveratrol has poor water solubility and low oral availability, which limits its application. Therefore, how to improve the water solubility of resveratrol and improve its bioavailability and stability is an important problem to be solved in the process of resveratrol utilization.

### SUMMARY

The disclosure provides a resveratrol composition and its preparation method. The composition is prepared by mixing resveratrol and poloxamer in proportion, which can improve the dissolution, bioavailability and stability of the resveratrol, and has good application and promotion prospects.

In order to achieve the above purpose, technical solutions of the present disclosure are specifically described as follows.

A resveratrol composition is prepared from following raw materials:
6%-60% by weight of resveratrol, and
40%-94% by weight of poloxamer.

Preferably, the poloxamer is poloxamer 407 or poloxamer 188.

Preferably, the resveratrol content is 10-20%.

The preparation method of the above resveratrol composition is selected from a grinding method or a melting preparation method.

The grinding method includes physically mixing evenly and then grinding or freezing grinding the resveratrol and poloxamer to form the resveratrol composition.

The melting preparation method includes heating the poloxamer to a molten state, then adding the resveratrol into the molten poloxamer in several times, stirring evenly to obtain a composition for standby, and then freezing grinding the composition for standby to prepare the resveratrol composition.

Preferably, a grinding equipment used in the grinding method includes a grinder, a ball mill or a freezing grinder.

A frequency of the grinder is 50-90 Hz, grinding times is 1-5 times, each grinding time is 30-180 min, and a grinding interval is 10-20 s.

A rotation speed of the ball mill is 200-400 r/min, each grinding time is 30-180 min, and grinding times is 1-5 times.

A freezing grinding temperature of the freezing grinder is −20-−50° C., a feeding frequency is 8-20 Hz, a grinding frequency is 10-60 Hz, and a fan speed is 30-80 Hz.

Preferably, a heating form of the melting preparation method is water bath heating or hot melt machine heating.

A water bath heating temperature is 60-80° C., a stirring speed is 100-300 r/min, and a stirring time is 60-80 min.

A heating temperature of the hot melt machine is 80-120° C., a stirring speed is 80-300 r/min, and a mixing time is 20-80 min.

A temperature of freezing grinding is −20-−50° C., a feeding frequency is 8-20 Hz, a grinding frequency is 30-80 Hz, and a fan speed is 30-80 Hz.

A preparation method of resveratrol composition microcapsules includes the following steps.

(1) The above resveratrol composition is taken, pure water, corn oil and Tween 80 are added, and a homogenization is performed to obtain a colostrum.

(2) A gelatin solution and an Arabic gum solution is mixed to obtain a mixture, which is added to the colostrum, and a homogenization is performed to obtain a multiple emulsion.

(3) The multiple emulsion is heated, the pH of the multiple emulsion is adjusted to 3-5, transglutaminase is added after cooling and stirred to obtain a dispersion.

(4) The pH of the dispersion is adjusted to 6.0, a freeze vacuum drying is performed after pre-freezing at −80° C. to obtain the resveratrol composition microcapsules.

Preferably, in step (1),

10-40 parts of the pure water, 20-50 parts of the corn oil and 1.5-5 parts of the Tween 80 are added to 1-2 parts of the resveratrol composition by weight;

the homogenization is performed at 20000-30000 r/min for 1-5 min.

In step (2),

the gelatin solution is prepared by adding 100-200 parts of pure water to 1-3 parts of gelatin by weight, and the Arabic gum solution is prepared by adding 100-200 parts of pure water to 1-3 parts of Arabic gum by weight;

a mass ratio of the gelatin, the Arabic gum and the resveratrol composition is (1-2): (1-2): (1-2);

the homogenization is performed at 8000-10000 r/min for 1-5 min.

Preferably, in step (3),

the multiple emulsion is placed in a constant temperature water bath at 35-45° C., and an acetic acid solution with a mass fraction of 15-25% is added to adjust the pH; after the temperature is reduced to below 15° C., the transglutaminase is added to obtain a mixture, and the mixture is stirred at 500-700 r/min for 1-2 h;

a mass ratio of the glutamine aminotransferase to the gelatin is 0.2: (1-2).

Preferably, in step (4),

a NaOH solution with a mass fraction of 15-25% is used to adjust the pH;

the pre-freezing is performed for 5-12 h;

a condition of the freeze vacuum drying is:

a vacuum degree is 1-50 Pa; a cold trap is −70-−80° C.; a temperature curve range is 24-34 h, and a heating plate temperature is −30-40° C.

An application of the above resveratrol composition in a preparation of resveratrol containing preparations is provided.

Preferably, the resveratrol containing preparations can be any acceptable dosage form such as powder, granule, tablet, capsule, soft capsule, soft candy, etc.

In summary, the present disclosure mixes the resveratrol and poloxamer in proportion to prepare a composition, and further improves the dissolution and bioavailability of the resveratrol in combination with microencapsulation technology. The preparation process is suitable for industrialization and has good application and promotion prospects.

### BRIEF DESCRIPTION OF THE DRAWINGS

FIG. 1 shows the water solubility of the resveratrol samples prepared with different raw material ratios.

FIG. 2 shows the water solubility of the resveratrol samples prepared by different processes.

FIG. 3 shows the change curve of plasma concentration within 4 h.

FIG. 4 shows the change curve of plasma concentration within 3 h.

FIG. 5 shows the particle size distribution of different samples.

Wherein, A is resveratrol raw material A, B is sample 3, C is sample 4, and D is sample 11.

### DETAILED DESCRIPTION OF THE EMBODIMENTS

Technical solutions in the embodiments of the present disclosure will be clearly and completely described below. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

The resveratrol raw materials used in the embodiments are as follows.

Resveratrol raw material A: 98% resveratrol Polygonum cuspidatum extract from Shaanxi JIAHERB Biotechnology Inc.;

Resveratrol raw material B: 98% resveratrol Polygonum cuspidatum extract from Xi'an ACETAR Biotechnology Inc.;

Resveratrol raw material C: 98% resveratrol Polygonum cuspidatum extract from Muhua Biotechnology Co., Ltd;

Resveratrol raw material D: 98% resveratrol Polygonum cuspidatum extract from Huirui Biotechnology Co., Ltd.

### Embodiment 1

Preparation of sample 1: 100 g of resveratrol raw material A and 900 g of poloxamer 407 were taken, mixed evenly, and then ground by a freezing grinder. The temperature of freezing grinding was −50° C., the feeding frequency was 10 Hz, the grinding frequency was 60 Hz, and the fan speed was 80 Hz. Sample 1 containing 10% resveratrol was obtained.

### Embodiment 2

Preparation of sample 1: 100 g of resveratrol raw material A and 400 g of poloxamer 407 were taken, mixed evenly, and then ground by a freezing grinder. The temperature of freezing grinding was −50° C., the feeding frequency was 10 Hz, the grinding frequency was 60 Hz, and the fan speed was 80 Hz. Sample 2 containing 20% resveratrol was obtained.

### Embodiment 3

Preparation of sample 1: 300 g of resveratrol raw material A and 200 g of poloxamer 407 were taken, mixed evenly, and then ground by a freezing grinder. The temperature of freezing grinding was −50° C., the feeding frequency was 10 Hz, the grinding frequency was 60 Hz, and the fan speed was 80 Hz. Sample 3 containing 60% resveratrol was obtained.

### Embodiment 4

Preparation of sample 4: 400 g of poloxamer 407 was taken, heated to a molten state in a 70° C. water bath, and 100 g of resveratrol A was added to the molten poloxamer 407 in several times, and stirred evenly. The stirring speed was 100 r/min and the stirring time was 80 min to obtain a composition for standby. The composition for standby was placed at room temperature and subjected to freezing grinding process. The temperature of freezing grinding was −40° C., the feeding frequency was 10 Hz, the grinding frequency was 50 Hz, and the fan speed was 80 Hz. Sample 4 containing 20% resveratrol was obtained.

### Embodiment 5

Preparation of sample 5: 2 g of the preparation of embodiment 3 was taken, 60 ml of pure water was added, 99 g of corn oil and 9.9 g of Tween 80 were added after dissolution, and the mixture was stirred with a homogenizer at 23000 r/min for 3 min to obtain a colostrum. 3 g of gelatin was taken and 300 g pure water was added, and 3 g Arabic gum was taken and 300 g pure water was added. After the two were mixed, they were added to the colostrum, and stirred with a homogenizer at 10000 r/min for 3 min to obtain a multiple emulsion. The multiple emulsion was kept at a constant temperature in a water bath at 40° C., and the pH was adjusted to 4.0 by adding 20% acetic acid solution. Then the temperature was reduced to below 15° C. in an ice bath, and 0.6 g of transglutaminase (TG) was added to solidify. The multiple emulsion was stirred with a magnetic stirrer at 600 r/min for 1 h, and then the pH was adjusted to 6.0 by NaOH solution with mass fraction of 20%. The dispersion was pre frozen in a refrigerator at −80° C. for 8 h and then subjected a freeze vacuum drying to obtain resveratrol microcapsules, which contained about 10% resveratrol.

The freeze vacuum drying conditions were:

vacuum degree: 1 Pa; cold trap −80° C.; temperature curve of heating plate: −30° C. during 0-3 h, −20° C. during 3-6 h, −10° C. during 6-9 h, 0° C. during 9-12 h, 10° C. during 12-15 h, 20° C. during 15-18 h, 30° C. during 18-21 h, 40° C. during 21-24 h.

### Embodiment 6

Preparation of sample 6: 4 g of the preparation of embodiment 3 was taken, 120 ml of pure water was added, 180 g of corn oil and 6.6 g of Tween 80 were added after dissolution, and the mixture was stirred with a homogenizer at 23000 r/min for 3 min to obtain a colostrum. 3 g of gelatin was taken and 100 g pure water was added, and 3 g Arabic gum was taken and 100 g pure water was added. After the two were mixed, they were added to the colostrum, and stirred with a homogenizer at 10000 r/min for 3 min to obtain a multiple emulsion. The multiple emulsion was kept at a constant temperature in a water bath at 40° C., and the pH was adjusted to 4.0 by adding 20% acetic acid solution. Then the temperature was reduced to below 15° C. in an ice bath, and 0.6 g of transglutaminase (TG) was added to solidify. The multiple emulsion was stirred with a magnetic stirrer at 600 r/min for 1 h, and then the pH was adjusted to 6.0 by NaOH solution with mass fraction of 20%. The dispersion was pre frozen in a refrigerator at −80° C. for 8 h and then subjected a freeze vacuum drying to obtain resveratrol microcapsules, which contained about 20% resveratrol.

The freeze vacuum drying conditions were:

vacuum degree: 1 Pa; cold trap −80° C.; temperature curve of heating plate: −30° C. during 0-3 h, −20° C. during 3-6 h, −10° C. during 6-9 h, 0° C. during 9-12 h, 10° C. during 12-15 h, 20° C. during 15-18 h, 30° C. during 18-21 h, 40° C. during 21-24 h.

### Embodiment 7

Preparation of sample 7: 400 g of resveratrol raw material A and 100g of poloxamer 407 were taken, mixed evenly, and then ground by a freezing grinder. The temperature of freezing grinding was −50° C., the feeding frequency was 10 Hz, the grinding frequency was 60 Hz, and the fan speed was 80 Hz. Sample 7 containing 80% resveratrol was obtained.

### Embodiment 8

Preparation of sample 8: 30 g of resveratrol raw material A and 470 g of poloxamer 407 were taken, mixed evenly, and then ground by a freezing grinder. The temperature of freezing grinding was −50° C., the feeding frequency was 10 Hz, the grinding frequency was 60 Hz, and the fan speed was 80 Hz. Sample 8 containing 6% resveratrol was obtained.

### Embodiment 9

Preparation of sample 11: 300 g of resveratrol raw material A and 200 g of poloxamer 188 were taken, mixed evenly, and then ground by a freezing grinder. The temperature of freezing grinding was −50° C., the feeding frequency was 8 Hz, the grinding frequency was 60 Hz, and the fan speed was 50 Hz. Sample 11 containing 60% resveratrol was obtained.

### Embodiment 10

Preparation of sample 12: 2 g of the preparation of embodiment 9 was taken, 20 mL of pure water was added, 33 g of corn oil and 3.3 g of Tween 80 were added after dissolution, and the mixture was stirred with a homogenizer at 23000 r/min for 3 min to obtain a colostrum. 3 g of gelatin was taken and 100 g pure water was added, and 3 g Arabic gum was taken and 100 g pure water was added. After the two were mixed, they were added to the colostrum, and stirred with a homogenizer at 10000 r/min for 3 min to obtain a multiple emulsion. The multiple emulsion was kept at a constant temperature in a water bath at 40° C., and the pH was adjusted to 4.0 by adding 20% acetic acid solution. Then the temperature was reduced to below 15° C. in an ice bath, and 0.6 g of transglutaminase (TG) was added to solidify. The multiple emulsion was stirred with a magnetic stirrer at 600 r/min for 1 h, and then the pH was adjusted to 6.0 by NaOH solution with mass fraction of 20%. The dispersion was pre frozen in a refrigerator at −80° C. for 8 h and then subjected a freeze vacuum drying to obtain resveratrol microcapsules, which contained about 10% resveratrol.

The freeze vacuum drying conditions were:

vacuum degree: 1 Pa; cold trap −80° C.; temperature curve of heating plate: −30° C. during 0-3 h, −20° C. during 3-6 h, −10° C. during 6-9 h, 0° C. during 9-12 h, 10° C. during 12-15 h, 20° C. during 15-18 h, 30° C. during 18-21 h, 40° C. during 21-24 h.

### Control Example 1

Preparation of sample 9: Resveratrol raw material A, glycerin monostearate and sodium carboxymethyl cellulose were weighed according to the mass ratio of 1:2:2, mixed evenly, dissolved with 5 times of absolute ethanol, concentrated under reduced pressure to volatilize the ethanol. After the mixture was viscous, freeze-dried at −20° C. and ground to obtain sample 9, which contained 20% resveratrol.

### Control Example 2

Preparation of sample 10: 100 g of resveratrol raw material A, 200 g of glycerin monostearate and 200 g of sodium carboxymethyl cellulose were taken, mixed evenly, and then ground by a freezing grinder. The temperature of freezing grinding was −50° C., the feeding frequency was 10 Hz, the grinding frequency was 60 Hz, and the fan speed was 80 Hz. Sample 6 containing 6% resveratrol was obtained.

### Experiment 1 Comparison of Water Solubility of Samples with Different Drug Loading

Samples 1, 2, 3, 7 and 8 prepared in embodiment 1, 2, 3, 7 and 8 (the theoretical content is 10%, 20%, 60%, 80% and 6%, respectively) were selected to determine the water solubility of the samples by high performance liquid chromatography.

Chromatographic conditions: Shimadzu LC-20AT high performance liquid chromatography; octadecylsilane bonded silica gel was used as filler; methanol and water (v/v=48:52) were used as mobile phases; the detection wavelength was 307 nm; the column temperature was 35° C., and the flow rate is 1 mL/min.

Preparation of control solution: a proper amount of resveratrol control sample was taken and accurately weighed, and the control solution containing 20 μg of resveratrol per 1 mL was prepared by adding methanol.

Preparation of test sample solution: about 1.00 g of the fine powder of each group of samples was taken, put into a conical flask with a stopper, accurately added with 50 mL of water, weighed, sonicated for 30 min, cooled, and weighed again. The lost weight was made up with water and shaken evenly. Then it was injected into the liquid chromatograph for 10 μL for measurement, and the test sample solution is obtained.

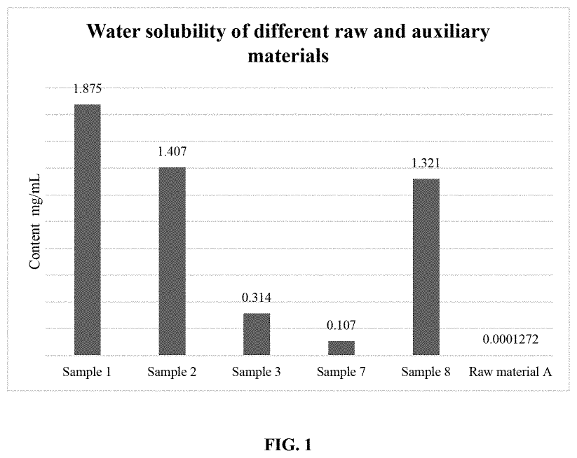

The experimental results are shown in FIG. 1 and Table 1. The samples with different raw material ratios prepared in embodiments 1, 2, 3, 7 and 8 can improve the water solubility of resveratrol. Among them, the water solubility of sample 3 was the highest, which was 1.875 mg/mL, followed by sample 2, which was 1.407 mg/mL, which was 14740 times higher than that of resveratrol raw material A.

TABLE 1

| Samples | Resveratrol content | Water solubility |
|---|---|---|
| Sample 1 | 10.00% | 1.875 mg/mL |
| Sample 2 | 20.00% | 1.407 mg/mL |
| Sample 3 | 60.00% | 0.314 mg/mL |
| Sample 7 | 80.00% | 0.107 mg/mL |
| Sample 8 | 6.00% | 1.321 mg/mL |
| Resveratrol raw | 98% | 0.0001272 mg/mL |
| material A | | |

### Experiment 2: Comparison of Water Solubility of Samples with the same Drug Loading and Different Processes

Samples 2, 4, 6, 9, and 10 prepared in embodiments 2, 4, 6, 9, and 10 (theoretical drug loading of 20%) were selected for water solubility determination by high performance liquid chromatography.

The chromatographic conditions, preparation of control solution and preparation of test sample solution were the same as those in the experiment 1.

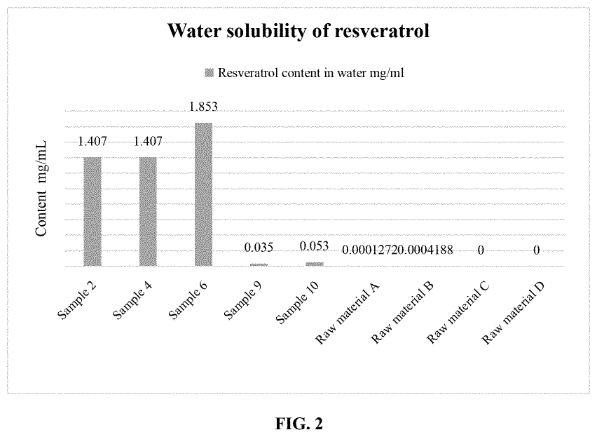

The experimental results are shown in FIG. 2 and table 2. The water solubility of samples 2, 4 and 6 was significantly higher than that of raw material samples, and was significantly higher than that of control samples 9 and 10. It can be seen that the water solubility of resveratrol was significantly improved by adding poloxamer freezing grinding, melting preparation and microencapsulation technology, and was better than that of sodium carboxymethyl cellulose and glycerin monostearate.

TABLE 2

| Samples | Resveratrol content | Water solubility |
|---|---|---|
| Sample 2 | 20.00% | 1.407 mg/mL |
| Sample 4 | 20.00% | 1.407 mg/mL |
| Sample 6 | 20.00% | 1.853 mg/mL |
| Sample 9 | 20.00% | 0.035 mg/mL |
| Sample10 | 20.00% | 0.053 mg/mL |
| Resveratrol raw material | 98% | 0.0001272 mg/mL |
| A | | |
| Resveratrol raw material | 98% | 0.0004188 mg/mL |
| B | | |
| Resveratrol raw material | 98% | Not detected |
| C | | |
| Resveratrol raw material | 98% | Not detected |
| D | | |

### Experiment 3 Bioavailability Experiment I

1. Experimental animals: SD (Sprague-Dawley) rats, male, 200 g, purchased from Shanghai SLAC experimental animal Co., Ltd.

2. Experimental grouping and administration

Dosage by gavage:

Sample 1 of test group 1: 1000 mg/kg (resveratrol content 10%);

Sample 2 of test group 2: 500 mg/kg (resveratrol content 20%);

Sample 5 of test group 3: 1000 mg/kg (resveratrol content 10%);

Sample 6 of test group 4: 500 mg/kg (resveratrol content 20%);

Sample 3 of test group 5: 166 mg/kg (resveratrol content 60%);

Resveratrol raw material A of test group 6: 100 mg/kg (resveratrol content 98.00%).

Except for the test group 6, samples of other groups were added into water to prepare suspension solution before administration. The test group 6 needed to be prepared into suspension solution with 0.5% sodium carboxymethyl cellulose (CMC-Na) aqueous solution. Before each gavage administration, the drug should be shaken well. The specific grouping is shown in Table 3.

| | | | Sample | Sample | Water/ | Dosing | Dosage |
|---|---|---|---|---|---|---|---|
| | Rat | Dosage | concentration | quantity/ | (CMC-Na) | volume/ | mg/ |
| Group | quantity | mg/kg | mg/mL | mg | mL | mL | piece |
| Blank | 3 | — | — | — | — | — | — |
| group | | | | | | | |
| Test | 6 | 1000 | 100 | 2000 | 20 | 2.5 | 250 |
| group 1 | | | | | | | |
| Test | 6 | 500 | 50 | 1000 | 20 | 2.5 | 125 |
| group 2 | | | | | | | |
| Test | 6 | 1000 | 100 | 2000 | 20 | 2.5 | 250 |
| group 3 | | | | | | | |
| Test | 6 | 500 | 50 | 1000 | 20 | 2.5 | 125 |
| group 4 | | | | | | | |
| Test | 6 | 166 | 16.6 | 332 | 20 | 2.5 | 41.5 |
| group 5 | | | | | | | |
| Test | 6 | 100 | 10 | 200 | 20 | 2.5 | 25 |
| group 6 | | | | | | | |

3. Sampling and sample processing:

Sampling: after gavage administration, blood samples were taken from the eyes of rats in each group at 5 min, 10 min, 15 min, 30 min, 1 h, 2 h and 4 h, and blood samples were collected with heparinized 1.5 mL EP tubes (or anticoagulant EP tubes). 0.5 mL blood was collected for each tube, and the number of blood collection tubes in the blank group was not less than 20.

Blood Sample Treatment:

(1) The test groups: □ The blood sample was subjected to centrifugation at 3000 rpm for 5 min at 4° C., and the upper plasma was taken. □ 200 μL of the plasma was taken, 1 mL of ethyl acetate was added and subject to vortex mix for 3 min to obtain a mixture. □ The mixture was centrifuged at 12000 rpm for 5 min, the supernatant was absorbed, and the ethyl acetate was blown dry.

(2) Blank group: The blood sample was subjected to centrifugation at 3000 rpm for 5 min, 150 μL of the upper plasma was taken and put into dry ice or ice for refrigeration.

4. HPLC analysis of resveratrol:

Chromatographic column: Inertsustain AQ-C18 (4.6×250 mm, 5 μm); mobile phase: acetonitrile: water=32:68 (v/v); flow rate: 1 mL/min; detection wavelength: 307 nm; sample size: 20 μL; column temperature: 30° C.

Treatment method of reference substance: Resveratrol standard was prepared into 1.0 μg/mL, 5.5 μg/mL, 10.0 μg/mL, 32.5 μg/mL, 55.0 μg/mL and 100.0 μg/mL resveratrol control solution. 10 μL of the above-mentioned control series solution was accurately sucked and added into 200 μL of plasma respectively, and the mixture was subject to vortex and shake for 30 s, then 1 mL of ethyl acetate was added and subjected to vortex and mix for 3 min. The mixture was centrifuged at 12000 rpm for 10 min, the supernatant was absorbed, and the ethyl acetate was dried at low temperature. 80 μL of the methanol was add, redissolved, vortexed for 3 min, centrifuged at 12000 r/min for 10 min, and 20 μL of supernatant was taken for HPLC analysis.

Treatment method of test sample: the blood samples of each group after treatment was taken and added with 80 μL of methanol for redissolution, vortex oscillation for 3 min and centrifugation at 12000 r/min for 10 min, and 20 μL of the supernatant was taken for HPLC analysis.

The standard curve was drawn according to the test results of the reference substance.

The actual concentration of each point of the standard was: 1.587 μg/mL, 3.175 μg/mL, 6.35 μg/mL, 25.4 μg/mL and 101.6 μg/mL. The standard curve was: Y=16560.7X+4943.75, R²=0.9997.

The concentration of resveratrol in plasma of each test sample at each time point was obtained by HPLC combined with the standard curve, and the bioavailability AUClast of each test sample was obtained by using Kinetica pharmacodynamics analysis software. The relative bioavailability between the samples of each embodiment and the bioavailability of resveratrol raw material A (JIAHERB) was calculated.

Relative bioavailability: F=AUCt·Dr/AUCr·Dt×100%

Where, t is test formulation and r is reference formulation.

AUCt is the bioavailability of samples in each embodiment, AUCr is the bioavailability of resveratrol raw material A.

Dt is the dosage of samples in each embodiment, and Dr is the dosage of resveratrol raw material A.

5. Relative bioavailability results

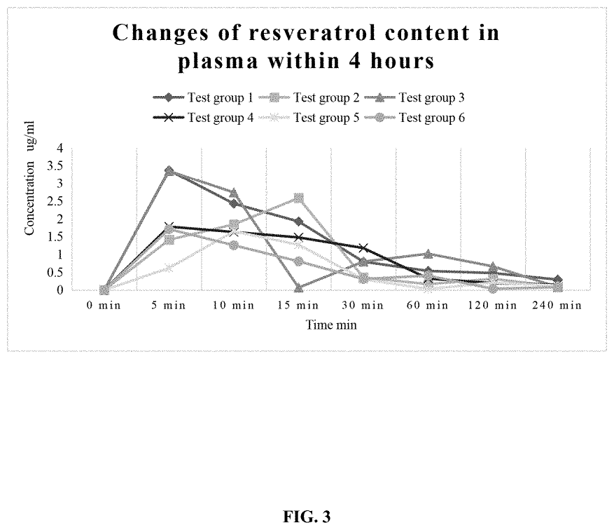

As shown in FIG. 3 and Table 4, both the freezing grinding process of resveratrol with poloxamer and the microcapsule preparation can effectively improve the relative bioavailability. When the content of resveratrol was the same, the effect of microencapsulation was better than that of freezing grinding. The bioavailability of 10% and 20% microencapsulated resveratrol samples reached 314.47% and 191.38% respectively, and the bioavailability of 10% and 20% freezing grinding process samples reached 323.45% and 182.87% respectively.

TABLE 4

| | AUClast | Dosage | Resveratrol | Relative |
|---|---|---|---|---|
| Group | (min*μg/mL ) | mg/kg | content | bioavailability |
| Test group 1 | 148.00 | 250 | 10.00% | 323.45% |
| Test group 2 | 83.6725 | 125 | 20.00% | 182.87% |
| Test group 3 | 143.891 | 250 | 10.00% | 314.47% |
| Test group 4 | 87.5683 | 125 | 20.00% | 191.38% |
| Test group 5 | 54.4652 | 41.5 | 60.00% | 119.51% |
| Test group 6 | 44.841 | 25 | 98.00% | 100.00% |

### Experiment 4 Bioavailability Experiment II

1. Experimental animals were the same as experiment 3.

2. Experimental grouping and administration

Dosage by gavage:

Sample 12 of test group 1: 1000 mg/kg (resveratrol content 10%);

Sample 11 of test group 2: 500 mg/kg (resveratrol content 20%);

Resveratrol raw material A of test group 3: 1000 mg/kg (resveratrol content 98.00%);

Samples of each group were added into water to prepare suspension solution before administration. Before each gavage administration, the drug should be shaken well. The specific grouping is shown in Table 5.

| | | | Sample | Sample | | Dosing | Dosage |
|---|---|---|---|---|---|---|---|
| | Rat | Dosage | concentration | quantity/ | Water/ | volume/ | mg/ |
| Group | quantity | mg/kg | mg/mL | mg | mL | mL | piece |
| Blank | 3 | — | — | — | — | — | — |
| group | | | | | | | |
| Test | 6 | 1000 | 100 | 2000 | 20 | 2.5 | 250 |
| group 1 | | | | | | | |
| Test | 6 | 166 | 16.6 | 332 | 20 | 2.5 | 41.5 |
| group 2 | | | | | | | |
| Test | 6 | 100 | 10 | 200 | 20 | 2.5 | 25 |
| group 3 | | | | | | | |

3. Sampling and sample processing:

Sampling: after gavage administration, blood samples were taken from the eyes of rats in each group at 2 min, 5 min, 10 min, 15 min, 30 min, 45 min, 1 h, 2 h and 3 h, and blood samples were collected with heparinized 1.5 mL EP tubes (or anticoagulant EP tubes). 0.5 mL blood was collected for each tube, and the number of blood collection tubes in the blank group was not less than 20.

Blood sample treatment was the same as experiment 3.

4. HPLC analysis of resveratrol:

Chromatographic column was the same as experiment 3.

Treatment method of reference substance was same as experiment 3.

Treatment method of test sample was the same as experiment 3.

The standard curve was drawn according to the test results of the reference substance.

The actual concentration of each point of the standard was: 0.3175 μg/ml, 0.635 μg/mL, 1.27 μg/mL, 2.54 μg/mL, 5.08 μg/mL and 10.16 μg/mL. The standard curve was: Y=128503x+6419.08, R²=0.998.

The concentration of resveratrol in plasma of each test sample at each time point was obtained by HPLC combined with the standard curve, and the bioavailability AUClast of each test sample was obtained by using Kinetica pharmacodynamics analysis software. The relative bioavailability between the samples of each embodiment and the bioavailability of resveratrol raw material A (Jiahe) was calculated.

Relative bioavailability: F=AUCt·Dr/AUCr·Dt×100%

Where, t is test formulation and r is reference formulation.

AUCt is the bioavailability of samples in each embodiment, AUCr is the bioavailability of resveratrol raw material A.

Dt is the dosage of samples in each embodiment, and Dr is the dosage of resveratrol raw material A.

5. Relative bioavailability results

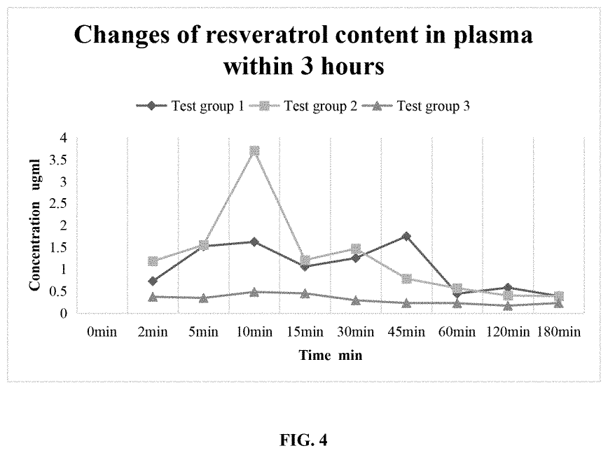

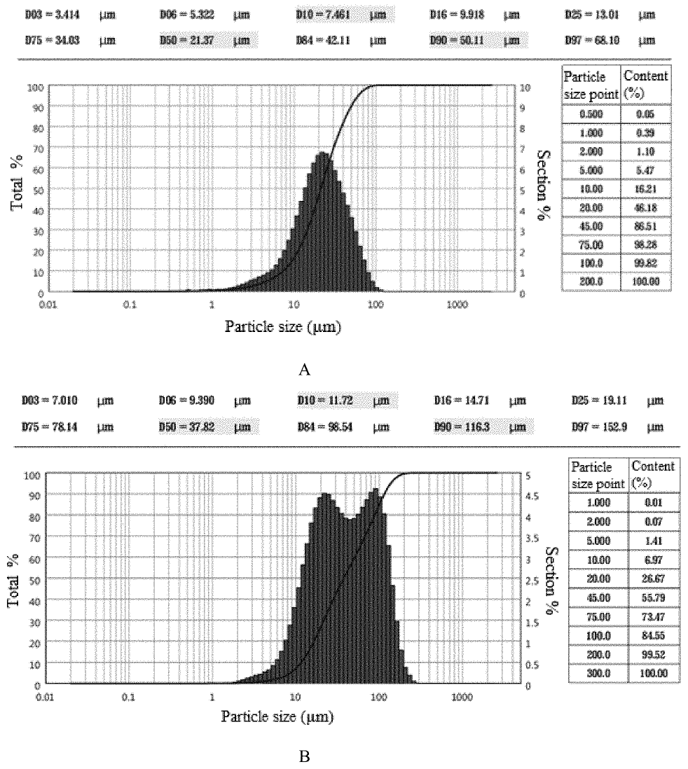

As shown in FIG. 4 and Table 6, the freezing grinding process of resveratrol with poloxamer 188 and the microcapsule preparation can effectively improve the relative bioavailability. The bioavailability of 60% freezing grinding samples reached 280.34%, and the bioavailability of 10% microencapsulated resveratrol samples reached 310.41%. Compared with the relative bioavailability experiment I, it can be found that for resveratrol raw material A, when the sample was dissolved for gavage, increasing CMC-Na affected and improved the bioavailability. In the freezing grinding process, the particle size of the sample was reduced and the bioavailability was improved.

TABLE 6

| | AUClast | Dosage | Resveratrol | Relative |
|---|---|---|---|---|
| Group | (min*μg/mL ) | mg/kg | content | bioavailability |
| Test group 1 | 129.20 | 1000 | 10.00% | 310.41% |
| Test group 2 | 117.14 | 166 | 60.00% | 280.34% |
| Test group 3 | 40.95 | 100 | 98.00% | 100% |

### Experiment 5 Particle Size Detection

The particle size of resveratrol raw material A, sample 3, sample 7 and sample 11 were detected by the Baite laser particle size detector.

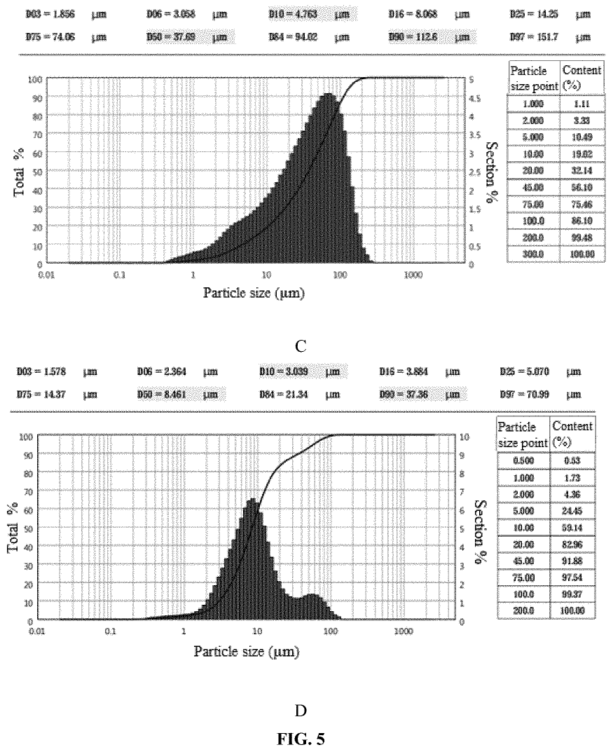

The results are shown in FIG. 5 and Table 7.

TABLE 7

| | Particle size distribution | | |
|---|---|---|---|
| Sample | D10 (μm) | D50 (μm) | D90 (μm) |
| Resveratrol | 7.461 | 21.37 | 50.11 |
| raw material A | | | |
| Sample 3 | 11.72 | 37.82 | 116.3 |
| Sample 4 | 4.763 | 37.69 | 112.6 |
| Sample 11 | 3.039 | 8.461 | 37.36 |

### Experiment 6 Stability Investigation

1. Test method

Packaging: inner sealed PE bag and outer sealed aluminum foil bag were used for packaging.

Test conditions: temperature 40° C.±2° C., humidity 75%±5% RH.

Test samples: sample 3 (10 g*15 bags) and sample 5 (10 g*15 bags).

Record: the appearance, content, moisture and ash of the samples taken in 0, 1, 2 and 3 months.

HPLC content detection method

□ Instrument parameters:

Chromatographic column: Inertsustain AQ-C18 (4.6×250 mm, 5 μm)

Mobile phase: acetonitrile: water=32:68 (v/v);

Flow rate: 1 mL/min;

Detection wavelength: 307 nm;

Sample size: 20 μL;

Column temperature: 30° C.

□ Treatment method of reference substance:

10 mg of the resveratrol reference substance was accurately weighed into a 50 mL volumetric flask, dissolved by adding about 20 mL of methanol, and then placed at room temperature. Methanol was used to volume up to the scale and shaken well. The resveratrol reference solution 200.0 μg/mL was obtained.

3. Preparation of test sample solution

10 mg of sample 3 test sample was accurately weighed into a 50 mL volumetric flask and dissolved by adding methanol at room temperature. The methanol was used to volume up to the scale and shaken well. The resveratrol reference solution 200.0 μg/mL was obtained after filtration.

10 mg of sample 5 test sample was accurately weighed into a 50 mL volumetric flask and dissolved by adding methanol at room temperature. The methanol was used to volume up to the scale and shaken well. The resveratrol reference solution 200.0 μg/mL was obtained after filtration.

Experimental Results

The results for Sample 3 are shown in Table 8, and the results for Sample 5 are shown in Table 9. In the accelerated 3-month stability test, the state and content of sample 3 and sample 5 were stable.

TABLE 8

| Item/month/result | 0 | 1 | 2 | 3 |
|---|---|---|---|---|
| Storage temperature | 40° C. | 40° C. | 40° C. | 40° C. |
| Storage RH | 75% RH | 75% RH | 75% RH | 75% RH |
| Appearance | Powder | Powder | Powder | Powder |
| Color | Standard | Standard | Standard | Standard |
| Smell | Standard | Standard | Standard | Standard |
| Resveratrol content | 60.69% | 60.90% | 60.61% | 60.55% |
| Water content | Standard | / | / | Standard |
| Total ash | Standard | / | / | Standard |

TABLE 9

| Item/month/result | 0 | 1 | 2 | 3 |
|---|---|---|---|---|
| Storage temperature | 40° C. | 40° C. | 40° C. | 40° C. |
| Storage RH | 75% RH | 75% RH | 75% RH | 75% RH |
| Appearance | Powder | Powder | Powder | Powder |
| Color | Standard | Standard | Standard | Standard |
| Smell | Standard | Standard | Standard | Standard |
| Resveratrol content | 10.98% | 10.49% | 11.01% | 10.89% |
| Water content | Standard | / | / | Standard |
| Total ash | Standard | / | / | Standard |

Various embodiments in the present specification are described in a progressive manner, and the emphasizing description of each embodiment is different from the other embodiments. The same and similar parts of various embodiments can be referred to for each other.

The above description of the disclosed embodiments enables those skilled in the art to realize or use the present disclosure. Many modifications to these embodiments will be apparent to those skilled in the art. The general principle defined herein can be realized in other embodiments without departing from the spirit or scope of the present disclosure. Therefore, the present disclosure will not be limited to these embodiments shown herein, but will conform to the widest scope consistent with the principle and novel features disclosed herein.

What is claimed is:

1. A resveratrol composition, wherein the resveratrol composition is prepared from following raw materials:
6% -60% by weight of resveratrol, and
40% -94% by weight of poloxamer.

2. The resveratrol composition of claim 1, wherein the poloxamer is poloxamer 407 or poloxamer 188.

3. A preparation method of the resveratrol composition of claim 1, wherein the method is selected from a grinding method or a melting preparation method;
the grinding method comprises: physically mixing evenly and then grinding or freezing grinding the resveratrol and poloxamer to form the resveratrol composition;
the melting preparation method comprises: heating the poloxamer to a molten state, adding the resveratrol into the molten poloxamer in several times, and stirring evenly to obtain a composition for standby; and then freezing grinding the composition for standby to prepare the resveratrol composition.

4. The preparation method of the resveratrol composition of claim 3, wherein a grinding equipment used in the grinding method comprises a grinder, a ball mill or a freezing grinder;
a frequency of the grinder is 50-90 Hz, grinding times is 1-5 times, each grinding time is 30-180 min, and a grinding interval is 10-20 s;
a rotation speed of the ball mill is 200-400 r/min, each grinding time is 30-180 min, and grinding times is 1-5 times;
a freezing grinding temperature of the freezing grinder is −20-−50° C., a feeding frequency is 8-20 Hz, a grinding frequency is 10-60 Hz, and a fan speed is 30-80 Hz.

5. The preparation method of the resveratrol composition of claim 3, wherein a heating form of the melting preparation method is water bath heating or hot melt machine heating;
a water bath heating temperature is 60-80° C., a stirring speed is 100-300 r/min, and a stirring time is 60-80 min;
a heating temperature of the hot melt machine is 80-120° C., a stirring speed is 80-300 r/min, and a mixing time is 20-80 min;
a temperature of freezing grinding is −20-−50° C., a feeding frequency is 8-20 Hz, a grinding frequency is 30-80 Hz, and a fan speed is 30-80 Hz.

6. A preparation method of resveratrol composition microcapsules, comprising:
(1) taking the resveratrol composition of claim 1, adding pure water, corn oil and Tween 80, and performing a homogenization to obtain a colostrum;
(2) mixing a gelatin solution and an Arabic gum solution to obtain a mixture, adding the mixture to the colostrum and performing a homogenization to obtain a multiple emulsion;
(3) heating the multiple emulsion, adjusting a pH of the multiple emulsion to 3-5, adding transglutaminase after cooling, and stirring to obtain a dispersion;
(4) adjusting a pH of the dispersion to 6.0, performing a freeze vacuum drying after pre-freezing at −80° C. to obtain the resveratrol composition microcapsules.

7. The preparation method of resveratrol composition microcapsules of claim 6, wherein
in step (1),
10-40 parts of the pure water, 20-50 parts of the corn oil and 1.5-5 parts of the Tween 80 are added to 1-2 parts of the resveratrol composition by weight;
the homogenization is performed at 20000-30000 r/min for 1-5 min;
in step (2),
the gelatin solution is prepared by adding 100-200 parts of pure water to 1-3 parts of gelatin by weight, and the Arabic gum solution is prepared by adding 100-200 parts of pure water to 1-3 parts of Arabic gum by weight;
a mass ratio of the gelatin, the Arabic gum and the resveratrol composition is (1-2): (1-2): (1-2);
the homogenization is performed at 8000-10000 r/min for 1-5 min.

8. The preparation method of resveratrol composition microcapsules of claim 6, wherein
in step (3),
the multiple emulsion is placed in a constant temperature water bath at 35-45° C., and an acetic acid solution with a mass fraction of 15-25% is added to adjust the pH;
after the temperature is reduced to below 15° C., the transglutaminase is added to obtain a mixture, and the mixture is stirred at 500-700 r/min for 1-2 h;
a mass ratio of the glutamine aminotransferase to the gelatin is 0.2: (1-2).

9. The preparation method of resveratrol composition microcapsules of claim 6, wherein
in step (4),
a NaOH solution with a mass fraction of 15-25% is used to adjust the pH;
the pre-freezing is performed for 5-12 h;
a condition of the freeze vacuum drying is:
a vacuum degree is 1-50 Pa; a cold trap is −70-−80° C.; a temperature curve range is 24-34 h, and a heating plate temperature is −30-40° C.
